(19) United States
(12) Patent Application Publication
(10) Pub. No.: US 2004/0115222 A1
(43) Pub. Date: Jun. 17, 2004

(54) Method for reduction of wrinkles

(76) Inventor: Michael Kane, New York, NY (US)

Correspondence Address: GOODWIN PROCTER L.L.P, 103 EISENHOWER PARKWAY, ROSELAND, NJ, 07068, US

(21) Appl. No.: 10/656,427

(22) Filed: Sep. 5, 2003

Related U.S. Application Data
(60) Provisional application No. 60/408,600, filed on Sep. 6, 2002.

(51) Int. Cl. A61K 39/08

(52) U.S. Cl. 424/239.1

(57) ABSTRACT

There is provided a method of reducing the appearance of facial wrinkles by repeatedly administering to a patient at defined time intervals a neurotoxin composition, such as BOTOX, said patient having been administered with an initial effective dosage of said neurotoxin composition based on said patient's diagnostic profile. The method comprises the step of, in accordance with a predefined administration schedule based on said patient's diagnostic profile and consisting of one or more time intervals, administering to said patient one or more incrementally decreasing amounts of said neurotoxin composition at each of said time intervals.

### CROSS-REFERENCE TO RELATED APPLICATIONS

This application claims the benefit of priority to U.S. Provisional Application No. 60/408,600, filed on Sep. 6, 2002, the contents of which are hereby incorporated by reference in the entirety.

### STATEMENT REGARDING FEDERALLY SPONSORED RESEARCH OR DEVELOPMENT

Not applicable.

### REFERENCES TO SEQUENCE LISTING, TABLES OR COMPUTER PROGRAM LISTING APPENDIX ON COMPACT DISK

Not applicable.

### BACKGROUND OF INVENTION

1. Field of the Invention

This invention generally relates to the methods of reducing wrinkles and improving the appearance of a patient by administration of a neurotoxin.

2. Description of Related Art

As patients age, the facial muscles may become hyperactive causing wrinkles and creases, for example, forehead creases, crow's feet or frown lines. The use of a neurotoxin to reduce such wrinkles and creases to improve the appearance of a patient were first described in the medical literature around 1992. A well-known type of neurotoxin used in this application is botulinum toxin type A. Allergan Inc. sells such a neurotoxin used for this purpose under the brand-name BOTOX®.

Typically, the neurotoxin is injected into the muscles at a dosage which eases the wrinkles and creases and lasts for several months. The problem with these injections when done in the described, traditional manner, is that the injections leave the patient with a total inability to frown for the first 2.5 to 3 months. As the effect of the injections diminishes, the patient begins to develop more and more motion resulting in completely normal motion by six to seven months after injection.

Performers, including newscasters, actors and actresses, and television personalities, cannot afford to look frozen. However, it is becoming increasingly difficult to get actors and actresses “of a certain age” who can emote on screen due to the popularity of BOTOX.

### SUMMARY OF THE INVENTION

There is provided a method of reducing the appearance of facial wrinkles by repeatedly administering to a patient at defined time intervals a neurotoxin composition, such as BOTOX, said patient having been administered with an initial effective dosage of said neurotoxin composition based on said patient's diagnostic profile. The method comprises the step of, in accordance with a predefined administration schedule based on said patient's diagnostic profile and consisting of one or more time intervals, administering to said patient one or more incrementally decreasing amounts of said neurotoxin composition at each of said time intervals.

Additional aspects, features and advantages of the present invention will become better understood with regard to the following description

### DETAILED DESCRIPTION OF THE INVENTION

What follows is a preferred embodiment of the present invention. It should be apparent to those skilled in the art that what is described herein is illustrative only and not limiting, having been presented by way of example only. All the features disclosed in this description may be replaced by alternative features serving the same purpose, and equivalents or similar purpose, unless expressly stated otherwise. Therefore, numerous other embodiments of the modifications thereof are contemplated as falling within the scope of the present invention as defined herein and equivalents thereto.

Patients have the least amount of lines during the first 2.5 to 3 months and think they look best during this period of time. Contrary to patient opinions, however, patients actually look better from 2.5 to 4.5 months after their injection. This is due to several factors. First, although they have slightly more wrinkles than during their first 2.5 months, these wrinkles are present during animation. In contrast, during the time after injections, patients are unable to appear animated because there are no wrinkles. Moreover, with the muscles still slightly weakened, and there are no wrinkles in repose. Also, with the muscles slightly weakened, their wrinkles during animation are also less than normal even though they appear to have a natural, normally expressive face.

Second, patients have not been able to animate for several months and thus the muscles beneath the skin have not been crinkling the skin like an accordion a few thousand times a day. During this rest period the skin actually repairs itself and the appearance of even deeply ingrained lines can lessen.

Decreasing dosages over a period of months allowed patients to have near normal expression. A problem though, is that this did not last as long. While doing injections this way allowed for a more normal expression and some of the benefits of the neuorotoxin, they did not reap the full benefit. That is because their skin never had a full holiday from motion and was not able to heal itself.

Often a performer has a hiatus or has had a break before going in front of the camera. To address the aforementioned deficiencies, one aspect of the present invention would be to first maximally inject the performer so that the performer's skin could recuperate. With a target date in mind the doses are decreased at a rate counterbalanced by an increase in their frequency. This way, the skin is maximally rested while more normal motion is achieved. Additionally, the benefits of the period of rest are maintained during a prolonged period of treatment.

An exemplary application of the present invention is as follows: a patient has a movie shoot in nine months: at nine months prior to shoot the patient is administered 30 units of neurotoxin into the glabella (frown lines), 20 units into the forehead and 25 units to the crow's feet area. Six months prior to the shoot, the patient is administered 25 units of neurotoxin into the glabella, 15 units into the forehead and 20 units to the crow's feet area. At three months prior to the shoot, the patient is administered 20 units of neurotoxin into the glabella, 12.5 units into the forehead and 15 units to the crow's feet area. Finally, at one month prior the shoot, the patient is administered 15 units into the glabella, 7.5 units into the forehead, 10 units to the crow's feet area.

### CONCLUSION

Having now described a preferred embodiment of the invention, it should be apparent to those skilled in the art that the foregoing is illustrative only and not limiting, having been presented by way of example only. All the features disclosed in this specification (including any accompanying claims, abstract, and drawings) may be replaced by alternative features serving the same purpose, and equivalents or similar purpose, unless expressly stated otherwise. Therefore, numerous other embodiments of the modifications thereof are contemplated as falling within the scope of the present invention as defined by the appended claims and equivalents thereto.

What is claimed is:

1. A method of reducing the appearance of facial wrinkles by repeatedly administering to a patient at defined time intervals a neurotoxin composition, said method comprising the steps of:
acquiring a diagnostic profile of said patient;
determining an initial effective dosage of said neurotoxin composition based on said patient's profile;
administering to said patient said initial effective dosage amount; and
in accordance with an administration schedule based on said patient's profile and consisting of one or more time intervals, administering to said patient at least one or more incrementally decreasing amounts of said neurotoxin composition at each of said time intervals.

2. The method as in claim 1 wherein said neurotoxin composition comprises BOTOX.

3. A method of reducing the appearance of facial wrinkles by repeatedly administering to a patient at defined time intervals a neurotoxin composition, said patient having been administered with an initial effective dosage of said neurotoxin composition based on said patient's diagnostic profile, said method comprising the step of:
in accordance with a predefined administration schedule based on said patient's diagnostic profile and consisting of one or more time intervals, administering to said patient at least one or more incrementally decreasing amounts of said neurotoxin composition at each of said time intervals.

4. The method as in claim 3 wherein said neurotoxin composition comprises BOTOX.

5. A method of generating a neurotoxin composition administration schedule for reducing the appearance of facial wrinkles by repeatedly administering to a patient at defined time intervals said neurotoxin composition, said method comprising the steps of:
acquiring a diagnostic profile of said patient;
determining an initial effective dosage of said neurotoxin composition based on said patient's profile;
determining said administration schedule based on said patient's profile and consisting of one or more time intervals to administer to said patient at least one or more incrementally decreasing amounts of said neurotoxin composition at each of said time intervals.

6. The method as in claim 5 wherein said neurotoxin composition comprises BOTOX.
